(19) United States
(12) Patent Application Publication
(10) Pub. No.: US 2015/0190054 A1
(43) Pub. Date: Jul. 9, 2015

(54) IMAGING APPARATUS FOR DIAGNOSIS AND IMAGE PROCESSING METHOD

(71) Applicant: TERUMO KABUSHIKI KAISHA, Tokyo (JP)

(72) Inventor: Kenji KANEKO, Yokohama-city (JP)

(73) Assignee: TERUMO KABUSHIKI KAISHA, Tokyo (JP)

(21) Appl. No.: 14/665,265

(22) Filed: Mar. 23, 2015

Related U.S. Application Data
(63) Continuation of application No. PCT/JP2012/006056, filed on Sep. 24, 2012.

(51) Int. Cl. A61B 5/00; A61B 8/12; A61B 5/02; A61B 8/00; A61F 2/82; A61B 19/00; A61B 8/08

(52) U.S. Cl. CPC A61B 5/0035; A61B 5/0066; A61B 19/5244; A61B 5/0084; A61B 8/12; A61B 8/0841; A61B 8/4416; A61F 2/82; A61B 5/02007; A61B 8/463; A61B 8/5207; A61B 8/5223; A61B 2019/5234; A61B 2019/528; A61B 2560/0437; A61B 2562/063

(57) ABSTRACT

An imaging apparatus is disclosed for diagnosis, which generates multiple horizontal cross-sectional images inside a body lumen, and a vertical cross-sectional image suitable for use by using the generated horizontal cross-sectional image. The imaging apparatus for diagnosis includes first extraction means for extracting a pixel corresponding to a cut position when a body lumen wall is cut by a plane substantially parallel to an axial direction, from a first horizontal cross-sectional image, second extraction means for extracting a pixel corresponding to a lumen surface when the body lumen wall is cut by the plane, from a second horizontal cross-sectional image, generation means for generating a vertical lumen image in such a way that a pixel obtained by projecting the pixel extracted by the second extraction means at the cut position is superimposed on the pixel extracted by the first extraction means, and display means for displaying the vertical lumen image.

### CROSS-REFERENCES TO RELATED APPLICATIONS

This application is a continuation of International Application No. PCT/JP2012/006056 filed on Sep. 24, 2012, the entire content of which is incorporated herein by reference.

### TECHNICAL FIELD

The disclosure generally relates to an imaging apparatus for diagnosis and an image processing method.

### BACKGROUND DISCUSSION

An imaging apparatus for diagnosis has been used for diagnosis of arteriosclerosis, preoperative diagnosis in performing endovascular treatment using a high-performance catheter such as a balloon catheter, a stent or the like, or for confirmation of postoperative results.

The imaging apparatus for diagnosis includes an intravascular ultrasound (IVUS) diagnosis apparatus and an optical coherence tomography (OCT) diagnosis apparatus, which respectively have different characteristics.

In addition, in recent years, an imaging apparatus for diagnosis (imaging apparatus for diagnosis which includes an ultrasound transceiver capable of transmitting and receiving ultrasound waves and an optical transceiver capable of transmitting and receiving light) which has an IVUS function and an OCT function in combination has also been proposed (for example, refer to JP-A-11-56752 and JP-T-2010-508973). According to this imaging apparatus for diagnosis, single scanning can generate both a cross-sectional image utilizing IVUS characteristics, which can enable measurement for a deep region, and a cross-sectional image utilizing OCT characteristics with a high resolution measurement.

Furthermore, if this imaging apparatus for diagnosis is applied, multiple cross-sectional images generated in the axial direction (cross-sectional image in a direction substantially orthogonal to an axis of a body lumen, hereinafter, referred to as a horizontal cross-sectional image) are processed. In this manner, it is possible to respectively generate and display a cross-sectional image in the vertical direction (axial direction of the body lumen) by using the IVUS function and a cross-sectional image in the vertical direction by using the OCT function. A cross-sectional image in a direction substantially parallel to the axis of the body lumen (hereinafter, referred to as a vertical cross-sectional image) which is generated and displayed in this way is effectively used in grasping an axial position or the like of a stent indwelled inside a blood vessel.

### SUMMARY

In accordance with an exemplary embodiment, it can be desirable to use a vertical cross-sectional image not only to grasp an axial position of a stent indwelled inside a blood vessel but also to perform more detailed analysis, for example, an analysis when the blood vessel is viewed just sideways. For example, a positional relationship between a gap of meshes of the stent and a bifurcated portion of the blood vessel can be analyzed by using the vertical cross-sectional image.

However, according to the conventional imaging apparatus for diagnosis, the vertical cross-sectional image is configured to display a cut surface when the blood vessel is vertically cut using a flat plane. Consequently, the above-described analysis cannot be performed since in order to perform the above-described analysis, both a cut surface of a blood vessel outer wall and a lumen surface of the blood vessel need to be displayed when the blood vessel is vertically cut using the flat plane.

For this reason, when the vertical cross-sectional image is generated and displayed, it can become relatively important to provide the vertical cross-sectional image by using an image mode suitable for various uses.

In accordance with an exemplary embodiment, an imaging apparatus for diagnosis is disclosed, which can generate multiple horizontal cross-sectional images inside a body lumen to generate a vertical cross-sectional image suitable for use by using the generated horizontal cross-sectional image.

In accordance with an exemplary embodiment, an imaging apparatus for diagnosis is disclosed, which can generate multiple first horizontal cross-sectional images and multiple second horizontal cross-sectional images which are cross-sectional images on a surface substantially orthogonal to an axial direction inside a body lumen, respectively in the axial direction by repeatedly transmitting and receiving a first signal and a second signal respectively while moving inside the body lumen in the axial direction. The imaging apparatus for diagnosis can include first extraction means for extracting a pixel corresponding to a cut position when a body lumen wall is cut by a plane substantially parallel to the axial direction, from the first horizontal cross-sectional image, second extraction means for extracting a pixel corresponding to a lumen surface when the body lumen wall is cut by the plane, from the second horizontal cross-sectional image, generation means for generating a vertical lumen image which is a lumen image when the body lumen wall is cut by the plane in such a way that a pixel obtained by projecting the pixel extracted by the second extraction means at the cut position is superimposed on the pixel extracted by the first extraction means, and display means for displaying the vertical lumen image generated by the generation means.

In accordance with an exemplary embodiment, an imaging apparatus for diagnosis is disclosed, which can generate multiple horizontal cross-sectional images inside a body lumen can be enabled to generate a vertical cross-sectional image suitable for use by using the generated horizontal cross-sectional image.

An image processing method of an imaging apparatus is disclosed for diagnosis which generates multiple first horizontal cross-sectional images and multiple second horizontal cross-sectional images which are cross-sectional images on a surface substantially orthogonal to an axial direction inside a body lumen, respectively in the axial direction by repeatedly transmitting and receiving a first signal and a second signal respectively while moving inside the body lumen in the axial direction, the method comprising: a first extraction step of extracting a pixel corresponding to a cut position when a body lumen wall is cut by a plane substantially parallel to the axial direction, from the first horizontal cross-sectional image; a second extraction step of extracting a pixel corresponding to a lumen surface when the body lumen wall is cut by the plane, from the second horizontal cross-sectional image; a generation step of generating a vertical lumen image which is a lumen image when the body lumen wall is cut by the plane in such a way that a pixel obtained by projecting the pixel extracted in the second extraction step at the cut position is superimposed on the pixel extracted in the first extraction step; and a display step of displaying the vertical lumen image generated in the generation step.

A non-transitory computer-readable recording medium with a program stored therein which causes a computer to execute each process of the image processing method as disclosed herein.

An imaging apparatus is disclosed for diagnosis which generates multiple first horizontal cross-sectional images and multiple second horizontal cross-sectional images which are cross-sectional images on a surface substantially orthogonal to an axial direction inside a body lumen, respectively in the axial direction by repeatedly transmitting and receiving a first signal and a second signal respectively while moving inside the body lumen in the axial direction, the apparatus comprising: first generation means for generating a planar image of a body lumen wall at a cut position when the body lumen wall is cut by a plane substantially parallel to the axis direction inside the body lumen, based on the multiple first horizontal cross-sectional images; second generation means for generating a three-dimensional image of a lumen surface of the body lumen which is interposed between the planar images, based on the multiple second horizontal cross-sectional images; and synthesis display means for displaying by synthesizing the planar image and the three-dimensional image.

Other characteristics and advantages of the disclosure will become apparent from the following description made with reference to the accompanying drawings. In the accompanying drawings, the same reference numerals are given to the same or similar configuration elements.

### BRIEF DESCRIPTION OF THE DRAWINGS

The accompanying drawings are incorporated in the description, configure a part of the description, represent embodiments of the imaging apparatus for diagnosis and an image processing method, and are used to describe principles of the imaging apparatus for diagnosis and an image processing method together with the description.

FIG. 1 is a view illustrating an external configuration of an imaging apparatus for diagnosis according to an exemplary embodiment of the disclosure.

FIG. 2 is a view illustrating an overall configuration of a probe unit and a cross-sectional configuration of a distal end portion.

FIG. 3A is a diagram illustrating a cross-sectional configuration of an imaging core.

FIG. 3B is a cross-sectional view taken along a plane substantially orthogonal to the rotation center axis as an ultrasonic transmitting and receiving position.

FIG. 3C is a cross-sectional view taken along a plane substantially orthogonal to the rotation center axis at an optical transmitting and receiving position.

FIG. 4 is a diagram illustrating a functional configuration of the imaging apparatus for diagnosis.

FIG. 5 is a diagram illustrating an example of a user interface of the imaging apparatus for diagnosis.

FIG. 6 is a view illustrating a generation process of a vertical cross-sectional image.

FIG. 7 is a view illustrating a generation process of a vertical lumen image.

FIG. 8 is a view illustrating the generation process of the vertical lumen image.

FIG. 9 is a flowchart illustrating flow in the generation process of the vertical lumen image.

FIG. 10A is a flowchart illustrating flow in a process of extracting a pixel for generating the vertical lumen image from an IVUS horizontal cross-sectional image.

FIG. 10B is a flowchart illustrating flow in a process of extracting a pixel for generating the vertical lumen image from an OCT horizontal cross-sectional image.

FIG. 11A is a view illustrating an example of the vertical lumen image.

FIG. 11B is a view illustrating an example of the vertical lumen image.

FIG. 12 is a view illustrating an example of the vertical lumen image.

FIG. 13 is a view illustrating a generation process of the vertical lumen image.

FIG. 14 is a view illustrating a generation process of the vertical lumen image.

### DETAILED DESCRIPTION

Hereinafter, each embodiment will be described in detail with reference to the accompanying drawings, when necessary.

1. External Configuration of Imaging Apparatus for Diagnosis

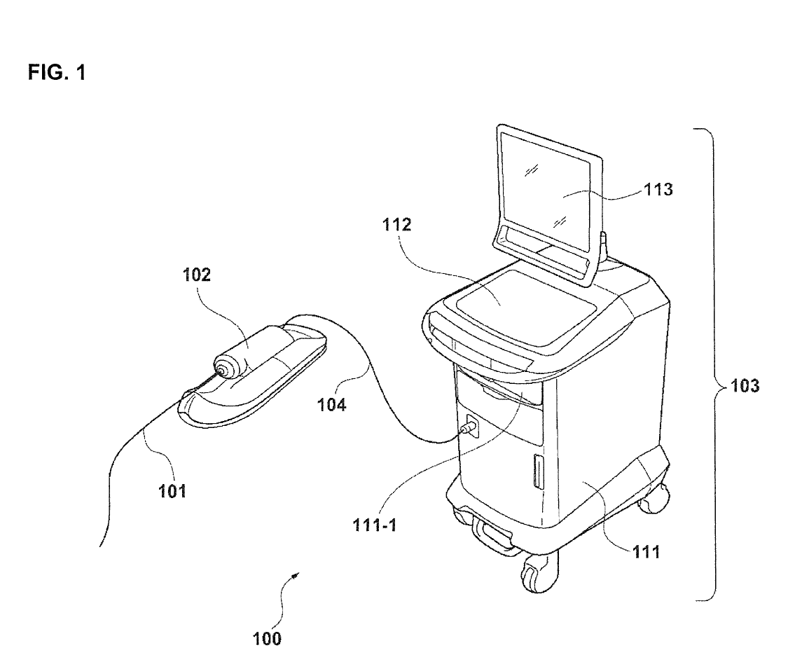

FIG. 1 is a view illustrating an external configuration of an imaging apparatus for diagnosis (imaging apparatus for diagnosis which includes an IVUS function and an OCT function) 100 according to an exemplary embodiment of the present disclosure.

As illustrated in FIG. 1, the imaging apparatus for diagnosis 100 can include a probe unit 101, a scanner and pull-back unit 102, and an operation control device 103. The scanner and pull-back unit 102 and the operation control device 103 can be connected to each other by a signal line 104 so that various signals can be transmitted.

The probe unit 101 has an internally inserted imaging core directly inserted into a body lumen and can include an ultrasound transceiver which transmits ultrasound waves into the body lumen based on a pulse signal and which receives reflected waves from the inside of the body lumen and an optical transceiver which continuously transmits transmitted light (measurement light) into the body lumen and which continuously receives reflected light from the inside of the body lumen. The imaging apparatus for diagnosis 100 measures a state inside the body lumen by using the imaging core.

The probe unit 101 can be detachably attached to the scanner and pull-back unit 102. A motor incorporated in the scanner and pull-back unit 102 is driven, thereby regulating an operation inside the body lumen in the axial direction and an operation inside the body lumen in the rotation direction of the imaging core, which is internally inserted into the probe unit 101. In addition, the scanner and pull-back unit 102 acquires the reflected wave received by the ultrasound transceiver and the reflected light received by the optical transceiver, and transmits the reflected wave and the reflected light to the operation control device 103.

The operation control device 103 can include a function for inputting various setting values upon each measurement, and a function for processing data obtained by the measurement and for displaying a cross-sectional image inside the body lumen (horizontal cross-sectional image and vertical cross-sectional image).

In the operation control device 103, the reference numeral 111 represents a main body control unit which generates ultrasound data based on the reflected waves obtained by the measurement, and which generates an ultrasound cross-sectional image by processing line data generated based on the ultrasound data. Furthermore, the main body control unit 111 generates interference light data by causing the reflected light obtained by the measurement to interfere with reference light obtained by separating the light from a light source, and generates an optical cross-sectional image by processing the generated line data based on the interference light data.

The reference numeral 111-1 represents a printer and DVD recorder, which prints a processing result in the main body control unit 111 or stores the processing result as data. The reference numeral 112 represents an operation panel, and a user inputs various setting values and instructions via the operation panel 112. The reference numeral 113 represents an LCD monitor as a display device, which displays a cross-sectional image generated in the main body control unit 111.

2. Overall Configuration of Probe Unit and Cross-Sectional Configuration of Distal End Portion

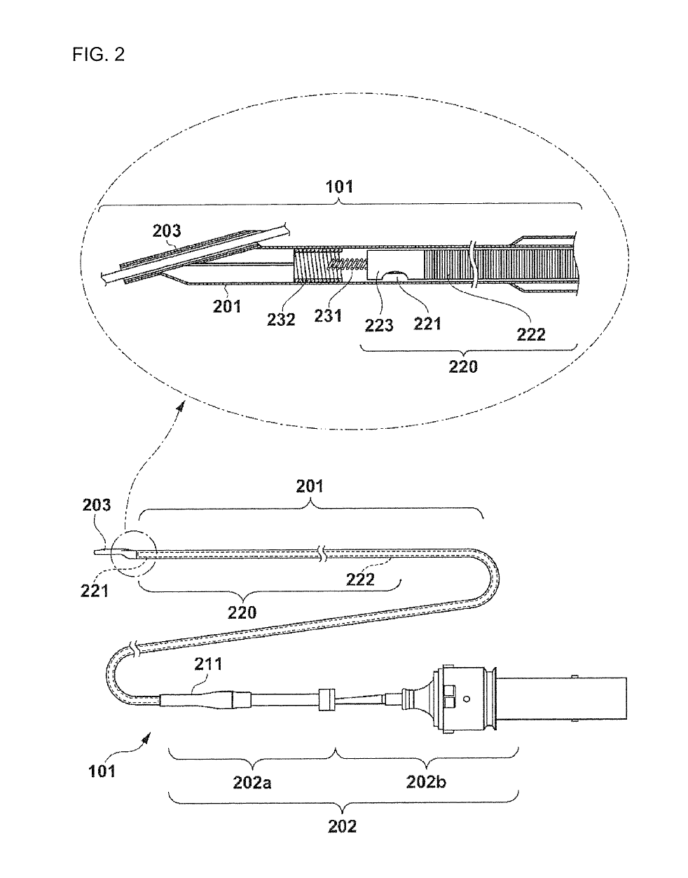

Next, an overall configuration of the probe unit 101 and a cross-sectional configuration of a distal end portion will be described with reference to FIG. 2. As illustrated in FIG. 2, the probe unit 101 is configured to include a long catheter sheath 201 to be inserted into the body lumen and a connector unit 202 to be arranged on the front side of a user, and which can be operated by the user without being inserted into the body lumen. The distal end of the catheter sheath 201 includes a tube 203 possessing a guide wire lumen configured to receive a guide wire. The catheter sheath 201 has a lumen, which is continuously formed from a connection portion with the guidewire lumen tube 203 to a connection portion with the connector unit 202.

An imaging core 220 which internally includes a transceiver 221 in which the ultrasound transceiver for transmitting and receiving the ultrasound waves and the optical transceiver for transmitting and receiving the light are arranged, and which includes a coil-shaped drive shaft 222 internally including an electrical signal cable and an optical fiber cable and transmitting rotary drive power for rotating the transceiver 221, which is inserted into the lumen of the catheter sheath 201 over substantially the entire length of the catheter sheath 201.

The connector unit 202 can include a sheath connector 202a configured to be integral with a proximal end of the catheter sheath 201, and a drive shaft connector 202b which is configured to rotatably fix the drive shaft 222 to the proximal end of the drive shaft 222.

An anti-kink protector 211 is disposed in a boundary section between the sheath connector 202a and the catheter sheath 201, which can help maintain a predetermined rigidity, and can help prevent bending (kinking) caused by a rapid change in physical properties.

The proximal end of the drive shaft connector 202b is detachably attached to the scanner and pull-back unit 102.

Next, the cross-sectional configuration of the distal end portion of the probe unit 101 will be described. The imaging core 220 can include a housing 223 having the transceiver 221 in which the ultrasound transceiver for transmitting and receiving the ultrasound waves and the optical transceiver for transmitting and receiving the light are arranged, and includes the drive shaft 222 for transmitting the rotary drive power for rotating the housing 223, which is inserted into the lumen of the catheter sheath 201 over substantially the entire length, thereby forming the probe unit 101.

The drive shaft 222 can cause the transceiver 221 to perform a rotary operation and an axial operation with respect to the catheter sheath 201, and has a property, which is flexible and can transmit rotation well. For example, the drive shaft 222 can be configured to have a multiplex and multilayer contact coil or the like formed of a metal wire such as a stainless steel wire or the like. Then, an electric signal cable and an optical fiber cable (optical fiber cable in a single mode) can be arranged inside the drive shaft 222.

The housing 223 has a shape in which a short cylindrical metal pipe partially has a cutout portion, and is formed by being cut out from a metal ingot, or is molded by means of metal powder injection molding (MIM), for example. In addition, an elastic member 231 having a short coil shape can be disposed on the distal end side of the housing 223.

The elastic member 231 is obtained by forming a stainless steel wire in a coil shape. The elastic member 231 is arranged on the distal end side, thereby help preventing the imaging core 220 from being caught on the inside of the catheter sheath 201 when the imaging core 220 is moved forward and rearward.

The reference numeral 232 represents a reinforcement coil, which is disposed in order to help prevent rapid bending of the distal end portion of the catheter sheath 201.

The guidewire lumen tube 203 has a guidewire lumen into which a guidewire can be inserted. The guidewire lumen tube 203 is used in receiving the guidewire inserted into the body lumen in advance and allowing the guidewire to guide the catheter sheath 201 to a lesion.

3. Cross-Sectional Configuration of Imaging Core

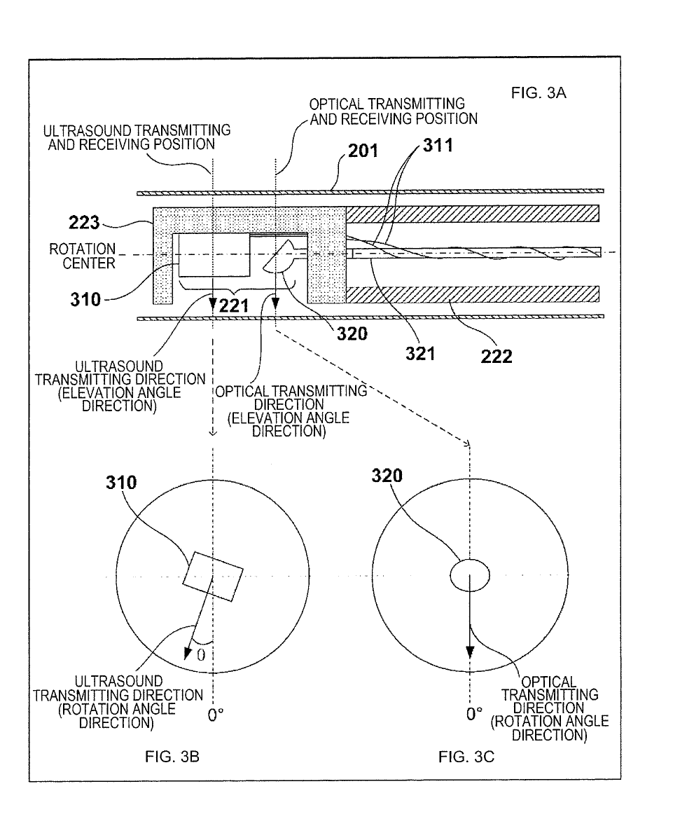

Next, a cross-sectional configuration of the imaging core 220 and an arrangement for the ultrasound transceiver and the optical transceiver will be described. FIGS. 3A-3C are diagrams illustrating the cross-sectional configuration of the imaging core, the arrangement for the ultrasound transceiver, and the optical transceiver, respectively.

As illustrated in FIG. 3A, the transceiver 221 arranged inside the housing 223 comprise an ultrasound transceiver 310 and an optical transceiver 320. The ultrasound transceiver 310 and the optical transceiver 320 are respectively arranged along the axial direction on the rotation center axis (on the one-dot chain line in FIG. 3A) of the drive shaft 222.

In accordance with an exemplary embodiment, the ultrasound transceiver 310 can be arranged on the distal end side of the probe unit 101, and the optical transceiver 320 can be arranged on the proximal end side of the probe unit 101.

In addition, the ultrasound transceiver 310 and the optical transceiver 320 are attached inside the housing 223 so that an ultrasound transmitting direction (elevation angle direction) of the ultrasound transceiver 310 and a light transmitting direction (elevation angle direction) of the optical transceiver 320 are respectively, for example, approximately 90° with respect to the axial direction of the drive shaft 222. In accordance with an exemplary embodiment, the ultrasound transceiver 310 can be attached to the optical transceiver 320 by causing each transmitting direction to be slightly deviated from 90° so as not to receive the reflection on a surface inside the lumen of the catheter sheath 201.

An electric signal cable 311 connected to the ultrasound transceiver 310 and an optical fiber cable 321 connected to the optical transceiver 320 are arranged inside the drive shaft 222. The electric signal cable 311 can be wound around the optical fiber cable 321 in a spiral shape.

FIG. 3B is a cross-sectional view taken along a plane substantially orthogonal to the rotation center axis at an ultrasound transmitting and receiving position. As illustrated in FIG. 3B, when a downward direction from the paper surface is zero degrees, the ultrasound transmitting and receiving direction (rotation angle direction (also referred to as an azimuth angle direction)) of the ultrasound transceiver 310 is θ degrees.

FIG. 3C is a cross-sectional view taken along a plane substantially orthogonal to the rotation center axis at an optical transmitting and receiving position. As illustrated in FIG. 3C, when the downward direction from the paper surface is zero degrees, the light transmitting and receiving direction (rotation angle direction) of the optical transceiver 320 is zero degrees. That is, the ultrasound transceiver 310 and the optical transceiver 320 are arranged so that the ultrasound transmitting and receiving direction (rotation angle direction) of the ultrasound transceiver 310 and the light transmitting and receiving direction (rotation angle direction) of the optical transceiver 320 are deviated from each other by θ degrees.

4. Functional Configuration of Imaging Apparatus for Diagnosis

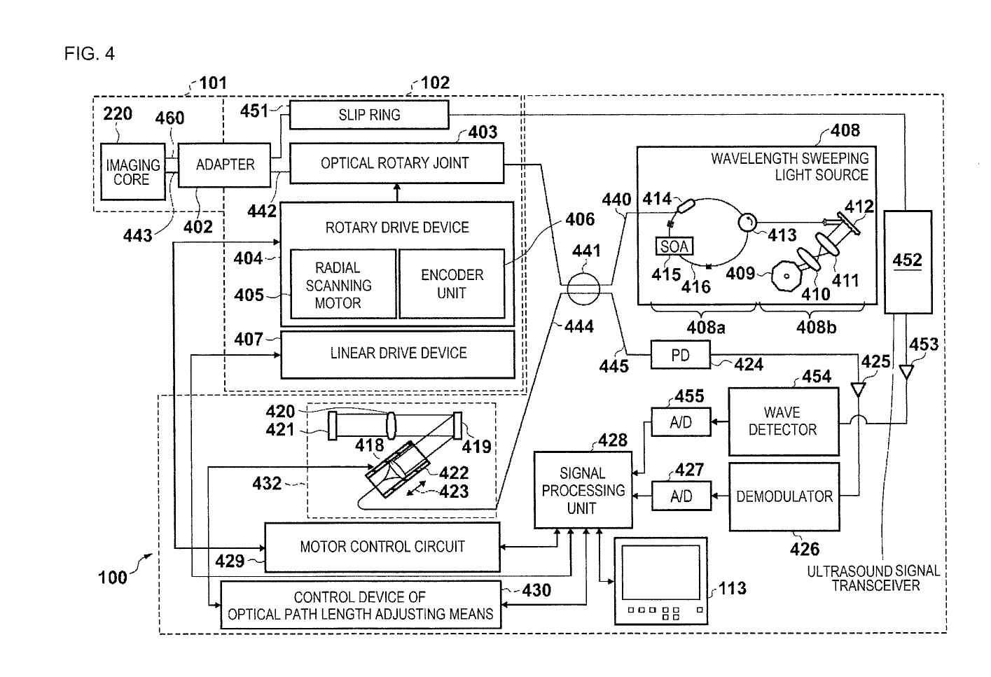

Next, a functional configuration of the imaging apparatus for diagnosis 100 will be described. FIG. 4 is a diagram illustrating the functional configuration of the imaging apparatus for diagnosis 100 which includes an IVUS function and an OCT function (here, a wavelength sweeping-type OCT as an example) in combination. An imaging apparatus for diagnosis including the IVUS function and other OCT functions in combination also has the same functional configuration. Therefore, description thereof will be omitted herein.

(1) IVUS Function

The imaging core 220 can include the ultrasound transceiver 310 inside the distal end of the imaging core 220. The ultrasound transceiver 310 can transmit ultrasound waves to biological tissues inside the body lumen while performing the rotary operation and the axial operation, based on pulse waves transmitted by an ultrasound signal transceiver 452, receives reflected waves (echoes) of the ultrasonic waves, and transmits the reflected waves to the ultrasound signal transceiver 452 as an ultrasound signal via an adapter 402 and a slip ring 451.

In the scanner and pull-back unit 102, a rotary drive portion side of the slip ring 451 is rotatably driven by a radial scanning motor 405 of a rotary drive device 404. Furthermore, a rotation angle of the radial scanning motor 405 is detected by an encoder unit 406. In addition, a linear drive device 407 can be arranged in the scanning and pull-back unit 102, and can regulate the axial operation of the imaging core 220 based on a signal from a signal processing unit 428.

The ultrasound signal transceiver 452 can include a transmitting wave circuit and a receiving wave circuit (not illustrated). The transmitting wave circuit transmits the pulse waves to the ultrasound transceiver 310 inside the imaging core 220 based on a control signal transmitted from the signal processing unit 428.

In addition, the receiving wave circuit receives an ultrasound signal from the ultrasound transceiver 310 inside the imaging core 220. The received ultrasound signal can be amplified by an amplifier 453, and then is input to and detected by a wave detector 454.

Furthermore, an A/D converter 455 generates digital data (ultrasound data) of one line by sampling the ultrasound signal output from the wave detector 454, for example, at 30.6 MHz by an amount of 200 points. Although 30.6 MHz is used here, this is calculated on the assumption that the sampling of 200 points is performed for a depth of 5 mm when sound velocity is set to 1530 m/sec. Therefore, the sampling frequency is not particularly limited thereto. Therefore, the sampling frequency is not particularly limited thereto.

The ultrasound data in units of lines, which is generated by the A/D converter 455 is input to the signal processing unit 428. The signal processing unit 428 converts the ultrasound data into a gray scale, thereby generating an IVUS horizontal cross-sectional image at each position in the axial direction inside the body lumen and outputting the ultrasound cross-sectional image to an LCD monitor 113 at a predetermined frame rate.

The signal processing unit 428 is connected to a motor control circuit 429, and receives a video synchronization signal of the motor control circuit 429. The signal processing unit 428 generates the IVUS horizontal cross-sectional image in synchronization with the received video synchronization signal.

In addition, the video synchronization signal of the motor control circuit 429 is also transmitted to the rotary drive device 404, and the rotary drive device 404 outputs a drive signal synchronized with the video synchronization signal.

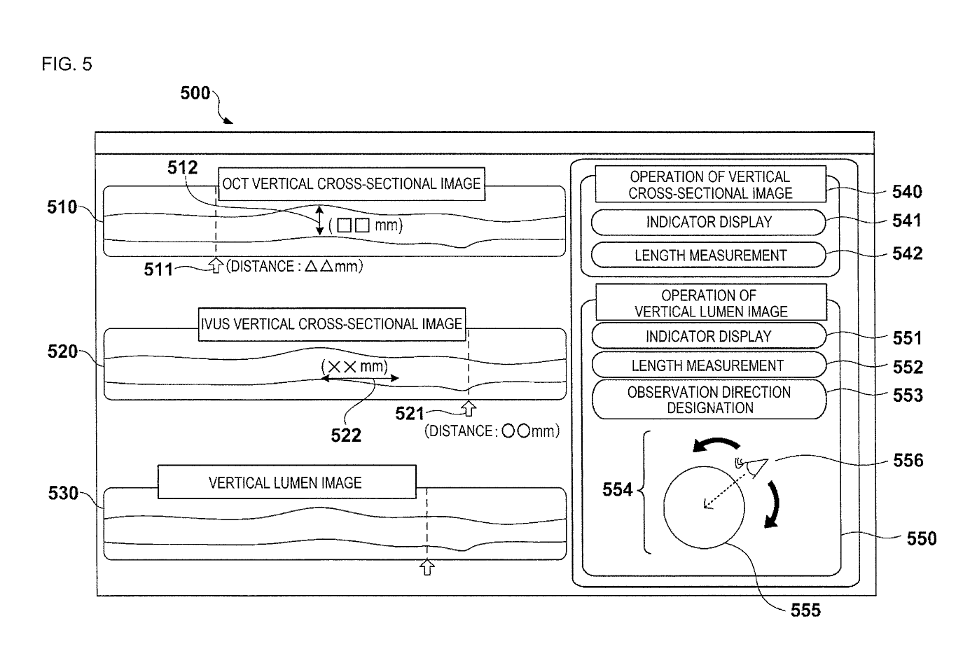

The above-described processing in the signal processing unit 428 and image processing relating to a user interface in the imaging apparatus for diagnosis 100 (to be described later with reference to FIGS. 5 to 13) can be realized in such a way that a predetermined program causes a computer to execute the processing in the signal processing unit 428.

(2) Function of Wavelength Sweeping-Type OCT

Next, a functional configuration of wavelength sweeping-type OCT will be described with reference to the same drawings. The reference numeral 408 represents a wavelength sweeping light source (swept laser), and is one type of an extended-cavity laser which can include an optical fiber 416 which is coupled to a semiconductor optical amplifier (SOA) 415 in a ring shape and a polygon scanning filter (408b).

Light output from the SOA 415 proceeds to the optical fiber 416, and enters the polygon scanning filter 408b. The light whose wavelength is selected here is amplified by the SOA 415, and is finally output from a coupler 414.

The polygon scanning filter 408b selects the wavelength in combination with a diffraction grating 412 for diffracting the light and a polygon mirror 409. In accordance with an exemplary embodiment, the light diffracted by the diffraction grating 412 can be concentrated on a surface of the polygon mirror 409 by two lenses (410 and 411). In this manner, only the light having a wavelength orthogonal to the polygon mirror 409 returns through the same optical path, and is output from the polygon scanning filter 408b. That is, time sweeping of the wavelength can be performed by rotating the polygon mirror 409.

For example, a 48-sided mirror can be used for the polygon mirror 409 whose rotation speed, can be, for example, approximately 50000 rpm. A wavelength sweeping system in which the polygon mirror 409 and the diffraction grating 412 can be combined with each other, which can enable high speed and high output wavelength sweeping.

The light of a wavelength sweeping light source 408 which is output from the coupler 414 is incident on one end (proximal end) of a first single mode fiber 440, and is transmitted to the distal end side of the first single mode fiber 440. The first single mode fiber 440 can be optically coupled to a second single mode fiber 445 and a third single mode fiber 444 in an optical coupler 441 located in the middle therebetween.

In accordance with an exemplary embodiment, on the further distal end side than the optical coupler 441 of the first single mode fiber 440, an optical rotary joint 403, which can transmit the light by coupling a fixed portion and a rotary drive unit to each other is disposed inside the rotary drive device 404.

Furthermore, a fifth single mode fiber 443 of the probe unit 101 can be detachably connected via the adapter 402 to the distal end side of a fourth single mode fiber 442 inside the optical rotary joint 403. In this manner, the light from the wavelength sweeping light source 408 can be transmitted to the fifth single mode fiber 443, which can be inserted into the imaging core 220 and can be rotatably driven.

The transmitted light is emitted from the optical transceiver 320 of the imaging core 220 to the biological tissues inside the body lumen while a rotary operation and an axial operation are performed. Then, the reflected light scattered on a surface or inside the biological tissues is partially captured by the optical transceiver 320 of the imaging core 220, and returns to the first single mode fiber 440 side through a rearward optical path. Furthermore, the light is partially transferred to the second single mode fiber 445 side by the optical coupler 441, and is emitted from one end of the second single mode fiber 445. Thereafter, the light is received by an optical detector (for example, a photodiode 424).

The rotary drive unit side of the optical rotary joint 403 is rotatably driven by the radial scanning motor 405 of the rotary drive device 404.

In accordance with an exemplary embodiment, a variable mechanism 432 having an optical path length for finely adjusting an optical path length of reference light can be disposed in the distal end opposite to the optical coupler 441 of the third single mode fiber 444.

In order for variations in the length of an individual probe unit 101 to be absorbed when the probe unit 101 is replaced and newly used, the variable mechanism 432 having this optical path length comprises optical path length changing means for changing an optical path length corresponding to the variations in the length.

The third single mode fiber 444 and a collimating lens 418 can be disposed on a one-axis stage 422 which is movable in an optical axis direction thereof as illustrated by an arrow 423, thereby forming the optical path length changing means.

In accordance with an exemplary embodiment, the one-axis stage 422 functions as the optical path length changing means having a variable enough range of the optical path length to absorb the variations in the optical path length of the probe unit 101 when the probe unit 101 is replaced. Furthermore, the one-axis stage 422 can also include an adjusting means for adjusting an offset. For example, even when the distal end of the probe unit 101 is not in close contact with the surface of the biological tissues, the one-axis stage can finely change the optical path length. In this manner, the optical path length can be set in a state of interfering with the reflected light from the surface position of the biological tissues.

The optical path length is finely adjusted by the one-axis stage 422. The light reflected on a mirror 421 via a grating 419 and a lens 420 is mixed with the light obtained from the first single mode fiber 440 side by the optical coupler 441 disposed in the middle of the third single mode fiber 444, and then is received by the photodiode 424.

Interference light received by the photodiode 424 in this way can be photoelectrically converted, and can be input to a demodulator 426 after being amplified by the amplifier 425. The demodulator 426 performs demodulation processing for extracting only a signal portion of the interference light, and an output therefrom is input to the A/D converter 427 as an interference light signal.

The A/D converter 427 performs sampling on the interference light signal, for example, at 90 MHz by an amount of 2048 points, and generates digital data (interference light data) of one line. In accordance with an exemplary embodiment, the reason for setting the sampling frequency to 90 MHz is on the assumption that approximately 90% of wavelength sweeping cycles (25.0 μsec) is extracted as the digital data of 2048 points, when a repetition frequency of the wavelength sweeping is set to 40 kHz. However, the sampling frequency is not particularly limited thereto.

The interference light data in the units of lines, which is generated by the A/D converter 427, is input to the signal processing unit 428. The signal processing unit 428 generates data in a depth direction (line data) by performing frequency resolution on the interference light data using the fast Fourier transform (FFT), and the data is subjected to coordinate transformation. In this manner, an OCT horizontal cross-sectional image is constructed at each position in the axial direction inside the body lumen, and is output to the LCD monitor 113 at a predetermined frame rate.

The signal processing unit 428 is further connected to a control device of optical path length adjusting means 430. The signal processing unit 428 controls a position of the one-axis stage 422 via the control device of optical path length adjusting means 430.

The processing related to a function of the wavelength sweeping-type OCT in the signal processing unit 428 can also be realized in such a way that a predetermined program causes a computer to execute the processing.

5. Description of User Interface

Next, a user interface displayed on the LCD monitor 113 will be described. FIG. 5 is a diagram illustrating an example of an exemplary user interface displayed on the LCD monitor 113. A cross-sectional image displayed on the user interface can include a horizontal cross-sectional image and a vertical cross-sectional image. However, hereinafter, a display of the vertical cross-sectional image (vertical cross-sectional image display screen 500) will be mainly described.

As illustrated in FIG. 5, the vertical cross-sectional image display screen 500 of the user interface can include an OCT vertical cross-sectional image display region 510 for displaying an OCT vertical cross-sectional image generated based on multiple OCT horizontal cross-sectional images generated in the axial direction inside the body lumen in the signal processing unit 428, an IVUS vertical cross-sectional image display region 520 for displaying an IVUS vertical cross-sectional image generated based on multiple IVUS horizontal cross-sectional images generated in the axial direction inside the body lumen in the signal processing unit 428, and a vertical cross-sectional image operation region 540 for operating the OCT vertical cross-sectional image and the IVUS vertical cross-sectional image.

If an “indicator display” button 541 is pressed down in the vertical cross-sectional image operation region 540, indicators 511 and 521 are respectively displayed in the OCT vertical cross-sectional image display region 510 and the IVUS vertical cross-sectional image display region 520. A user can move the displayed indicators 511 and 521 in the rightward direction or in the leftward direction of the vertical cross-sectional image display screen 500 by using an operation device, for example, a mouse, or a trackball on the operation panel 112. In this manner, the user can recognize each position (distance) in the axial direction inside the body lumen.

In addition, if a “length measurement” button 542 is pressed down in the vertical cross-sectional image operation region 540, length measurement instruments 512 and 522 for measuring any desired length are respectively displayed in the OCT vertical cross-sectional image display region 510 and the IVUS vertical cross-sectional image display region 520. The user can measure the length of a measurement target length by aligning an end point of the length measurement instruments 512 and 522 with the measurement target using the operation device such as the mouse, or the trackball on the operation panel 112.

Furthermore, a vertical lumen image display region 530 for displaying a vertical lumen image (details to be described later) generated based on the multiple OCT horizontal cross-sectional images and the multiple IVUS horizontal cross-sectional images which are generated in the axial direction inside the body lumen, and a vertical lumen image operation region 550 for operating the vertical lumen image are displayed on the vertical cross-sectional image display screen 500 of the user interface.

In the vertical lumen image operation region 550, an “indicator display” button 551 and a “length measurement” button 552 are the same as the “indicator display” button 541 and the “length measurement” button 542 in the vertical cross-sectional image operation region 540. Thus, here, description thereof will be omitted.

If an “observation direction designation” button 553 is pressed down in the vertical lumen image operation region 550, the user can change an observation direction of the vertical lumen image displayed in the vertical lumen image display region 530.

The reference number 554 is a view illustrating the observation direction of the vertical lumen image, and the reference numeral 555 schematically illustrates the horizontal cross-sectional image of the body lumen. In addition, the reference numeral 556 schematically illustrates an observation position. In an example of FIG. 5, the vertical lumen image when viewed in the direction indicated by a dotted arrow is displayed in the vertical lumen image display region 530. The observation position 556 can be changed along a thick arrow (that is, along a horizontal cross-sectional image 555, in the circumferential direction). The observation position 556 can be moved in the circumferential direction, thereby reconstructing the vertical lumen image to be displayed in the vertical lumen image display region 530.

In accordance with an exemplary embodiment, in this way, various types of vertical cross-sectional images can be displayed on the vertical cross-sectional image display screen 500. Accordingly, the user can perform various analyses by using the respective vertical cross-sectional images. For example, when the imaging apparatus for diagnosis 100 is used in diagnosing conditions inside the blood vessel, the blood vessel outer wall (body lumen wall) can be drawn on the IVUS vertical cross-sectional image. Accordingly, a shape change in the blood vessel outer wall in the axial direction can be grasped or understood. In addition, an upper cross section and a lower cross section of the stent can be drawn on the OCT vertical cross-sectional image. Accordingly, whether the stent is arranged along the blood vessel outer wall in the axial direction or is arranged at a suitable position in the axial direction can be grasped or understood.

Furthermore, in a case of the vertical lumen image, meshes of the stent and a bifurcated portion of the blood vessel can be drawn on the vertical lumen image. Accordingly, a relationship between a position of a gap in the meshes of the stent and a position of the bifurcated portion of the blood vessel can be grasped or understood.

That is, according to the imaging apparatus for diagnosis 100, it is possible to generate and display the vertical cross-sectional image by using a display mode suitable for respective uses.

6. Description of Vertical Cross-Sectional Image

Next, a generation method of the OCT vertical cross-sectional image, the IVUS vertical cross-sectional image, and the vertical lumen image which are displayed on the vertical cross-sectional image display screen 500 will be described. Hereinafter, a case will be described where the imaging apparatus for diagnosis 100 is used in diagnosing conditions inside the blood vessel.

6.1 Generation Method of OCT Vertical Cross-Sectional Image and IVUS Vertical Cross-Sectional Image

First, the generation method of the OCT vertical cross-sectional image and the IVUS vertical cross-sectional image will be described. The generation method of the OCT vertical cross-sectional image and the generation method of the IVUS vertical cross-sectional image are basically the same as each other. Thus, here, the generation method of the OCT vertical cross-sectional image will be described.

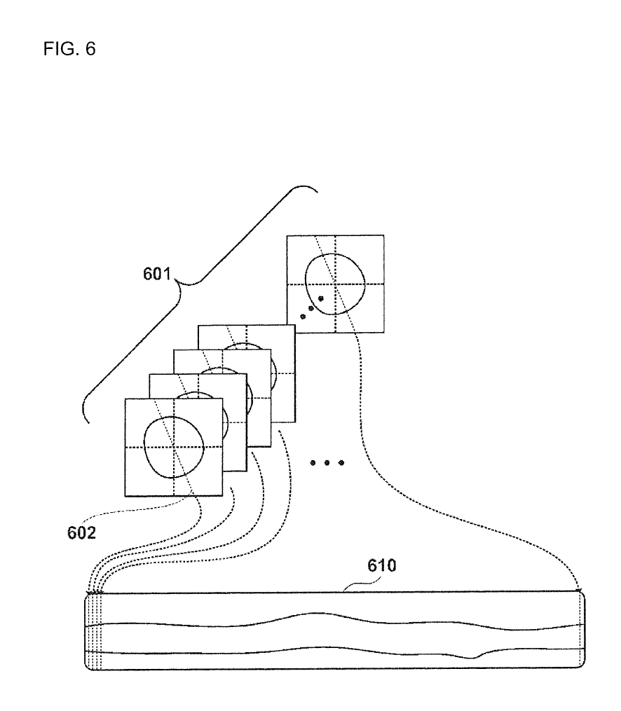

FIG. 6 is a view for illustrating the generation method of the OCT vertical cross-sectional image. The reference numeral 601 in FIG. 6 illustrates multiple OCT horizontal cross-sectional image groups generated in the axial direction inside the body lumen in the signal processing unit 428. The reference numeral 602 illustrates a cut position for generating the OCT vertical cross-sectional image. The cut position 602 is a position indicating a cut surface when the blood vessel outer wall is cut by a plane substantially parallel to the axial direction, and is regulated by a straight line passing through the image center of the OCT horizontal cross-sectional image. In an example of FIG. 6, the cut position 602 is regulated to be located at a position, which is rotated by a predetermined angle around the axis in the vertical direction of the OCT horizontal cross-sectional image.

The reference numeral 610 is the OCT vertical cross-sectional image, and each pixel vertically arrayed at each position in the axial direction corresponds to each pixel vertically arrayed on the cut position 602 of the OCT horizontal cross-sectional image at the corresponding position.

In accordance with an exemplary embodiment, the OCT vertical cross-sectional image can be generated by reading out the OCT horizontal cross-sectional image group 601 included in a predetermined range in the axial direction, by extracting each pixel at the cutting position 602 from each OCT horizontal cross-sectional image, and by arranging each pixel at the corresponding position in the axial direction.

6.2 Description of Vertical Lumen Image

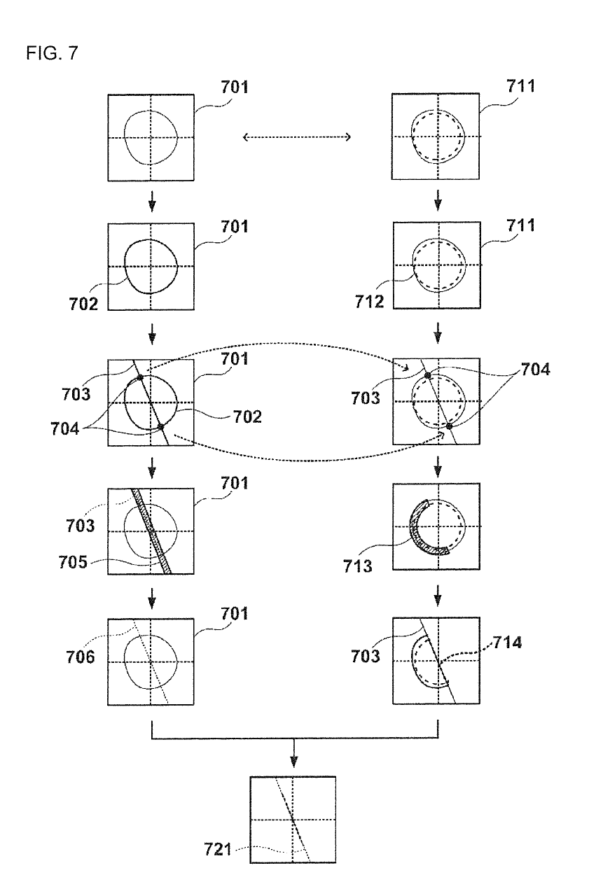

Next, the generation method of the vertical lumen image will be described. FIG. 7 is a view for illustrating the generation method of the vertical lumen image. In FIG. 7, the left side of the FIG. 7 illustrates the IVUS horizontal cross-sectional image generated in the signal processing unit 428, and the right side of FIG. 7 illustrates the OCT horizontal cross-sectional image generated in the signal processing unit 428.

An IVUS horizontal cross-sectional image 701 and an OCT horizontal cross-sectional image 711 are configured to respectively have the same position in the axial direction. First, position alignment for both images is performed.

Subsequently, an extraction process for extracting the blood vessel outer wall is performed on the IVUS horizontal cross-sectional image 701. The reference numeral 702 illustrates the blood vessel outer wall extracted from the IVUS horizontal cross-sectional image 701.

Next, the cut position 703 is superimposed on the IVUS horizontal cross-sectional image 701 from which the blood vessel outer wall is extracted so as to obtain an intersection point 704 between the cut position 703 and the blood vessel outer wall 702. The cut position 703 is a position indicating a cut surface when the blood vessel outer wall is cut by a plane substantially parallel to the axial direction, and is a straight line which has an angle substantially orthogonal to an angle around the axis which is designated by the observation position 556 in the vertical lumen image operation region 550, and which passes through the image center of the IVUS horizontal cross-sectional image 701. The intersection point 704 is used when the OCT horizontal cross-sectional image 711 is processed.

Next, each pixel included in a predetermined width region 705 including the cut position 703 is extracted from the IVUS horizontal cross-sectional image 701 on which the cut position 703 is specified. Furthermore, each extracted pixel is projected to the cut position 703, thereby extracting each pixel 706 at the cut position 703 to which the pixel is projected.

In accordance with an exemplary embodiment, an extraction process for extracting a stent position is first performed on the OCT horizontal cross-sectional image 711. The reference numeral 712 illustrates the stent position extracted from the OCT horizontal cross-sectional image 711.

Next, the cut position 703 and the intersection point 704 are superimposed on the OCT horizontal cross-sectional image 711 from which a stent position 712 is extracted. A predetermined width semicircle region 713 in which a distance between the intersection points 704 serves as a diameter and the intersection point 704 serves as an end point, and which includes the stent position is generated, thereby extracting each pixel included in the semicircle region 713. Furthermore, each extracted pixel is projected to the cut position 703, thereby extracting each pixel 714 at the cut position 703 to which the pixel is projected.

Finally, each pixel 714 extracted from the OCT horizontal cross-sectional image 711 is superimposed on each pixel 706 extracted from the IVUS horizontal cross-sectional image 701, thereby generating a pixel 721 for generating the vertical lumen image.

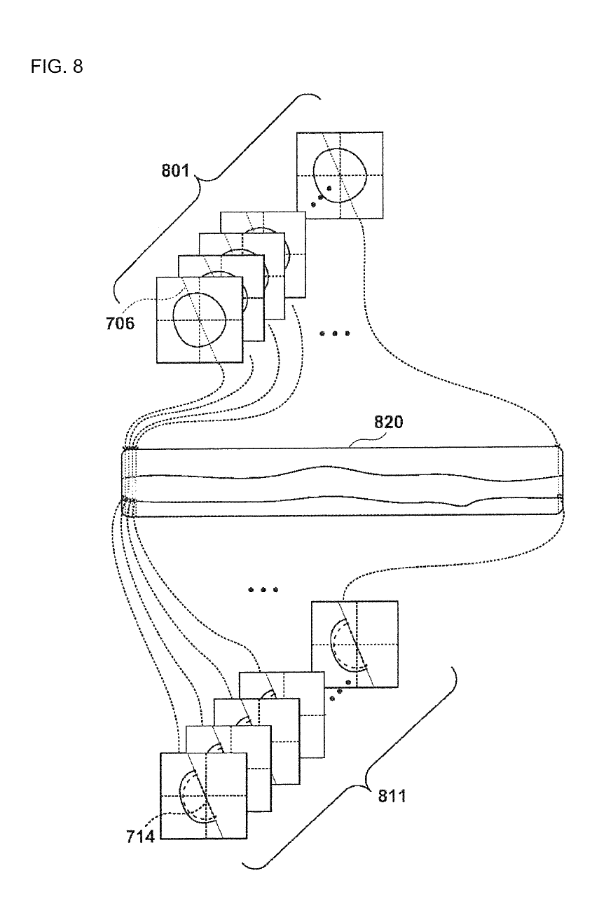

FIG. 8 is a view for illustrating a method of generating the vertical lumen image, based on the IVUS horizontal cross-sectional image 701 and the OCT horizontal cross-sectional image 711. In FIG. 8, the reference numeral 801 illustrates a group of the IVUS horizontal cross-sectional images 701 in which each pixel 706 is projected at the cut position. In addition, the reference numeral 811 illustrates a group of the OCT horizontal cross-sectional images 711 in which each pixel 714 is projected at the cut position.

The reference numeral 820 is a vertical lumen image, and each pixel vertically arrayed at each position in the axial direction is generated by superimposing each pixel 714 vertically arrayed at the cut position 703 of the OCT horizontal cross-sectional images 711 at the corresponding position on each pixel 706 vertically arrayed at the cut position 703 of the IVUS horizontal cross-sectional images 701 at the corresponding position (that is, generated by arranging the pixel 721 at the corresponding position in the axial direction).

7. Flow in Generation Process of Vertical Lumen Image

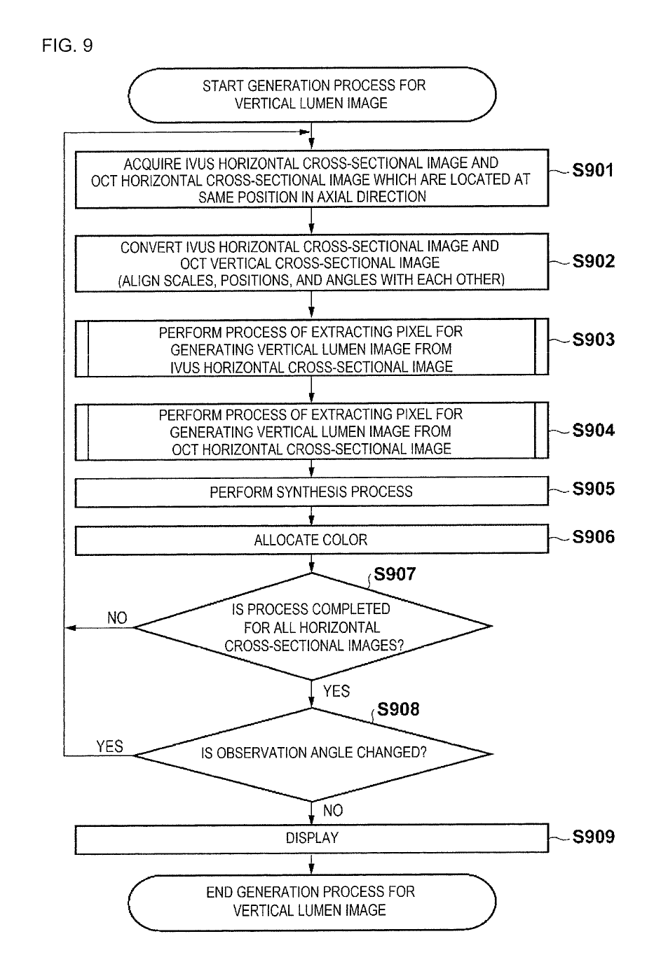

Next, flow in the generation process of the vertical lumen image will be described with reference to FIGS. 9 to 11B.

7.1 Overall Flow in Generation Process of Vertical Lumen Image

FIG. 9 is a view illustrating overall flow in the generation process of the vertical lumen image, and the generation process is performed in the signal processing unit 428.

In Step S901, the IVUS horizontal cross-sectional image and the OCT horizontal cross-sectional image, which are located at the same position in the axial direction, are acquired from multiple horizontal cross-sectional images included in a predetermined range in the axial direction inside the body lumen.

In Step S902, a conversion process is performed on the IVUS horizontal cross-sectional image and the OCT horizontal cross-sectional image, thereby causing both images to have aligned scales, positions, and angles.

In Step S903, the pixel 706 for generating the vertical lumen image is extracted from the IVUS horizontal cross-sectional image. In Step S904, the pixel 714 for generating the vertical lumen image is extracted from the OCT horizontal cross-sectional image. Processes in Steps S903 and S904 will be described in detail later.

In Step S905, a synthesis process for generating the pixel 721 is performed by superimposing the pixel 714 extracted in Step S904 on the pixel 706 extracted in Step S903. In Step S906, a color is allocated to the pixel 721 generated in Step S905. The color allocation is performed on a region to be emphasized depending on use of the vertical lumen image. For example, if a user wants to grasp a position of meshes of the stent, the position is emphasized by allocating a predetermined color (for example, a red color) to the position. Alternatively, if the user wants to grasp a position of the bifurcated portion of the blood vessel, the position can be emphasized by allocating a predetermined color (for example, a black color) to the position.

In Step S907, it is determined whether or not the above-described process is performed for all of the horizontal cross-sectional images included in a predetermined range in the axial direction inside the body lumen. When it is determined that the process is not performed, the procedure returns to Step S901 to continue the process.

In accordance with an exemplary embodiment, when it is determined that the above-described process is performed for all of the horizontal cross-sectional images in Step S907, the procedure proceeds to Step S908. In Step S908, it can be determined whether or not an instruction to change an observation angle is input on the vertical cross-sectional image display screen 500.

In Step S908, when it is determined that the instruction to change the observation angle is not input, the procedure proceeds to Step S909 so as to display the vertical lumen image which is generated by the processes in Steps S901 to S907 and to which the color is allocated. Thereafter, the generation process of the vertical lumen image ends.

In accordance with an exemplary embodiment, in Step S908, when it is determined that the instruction to change the observation angle is input, the cut position orthogonal to the changed observation angle is calculated. Then, the processes in Steps S901 to S907 can be performed by using the calculated cut position. Then, the vertical lumen image display region 530 is updated from the vertical lumen image which is generated by the processes in Steps S901 to S907 and to which the color is allocated. Thereafter, the generation process of the vertical lumen image ends.

7.2 Details of Process of Extracting Pixel for Generating Vertical Lumen Image

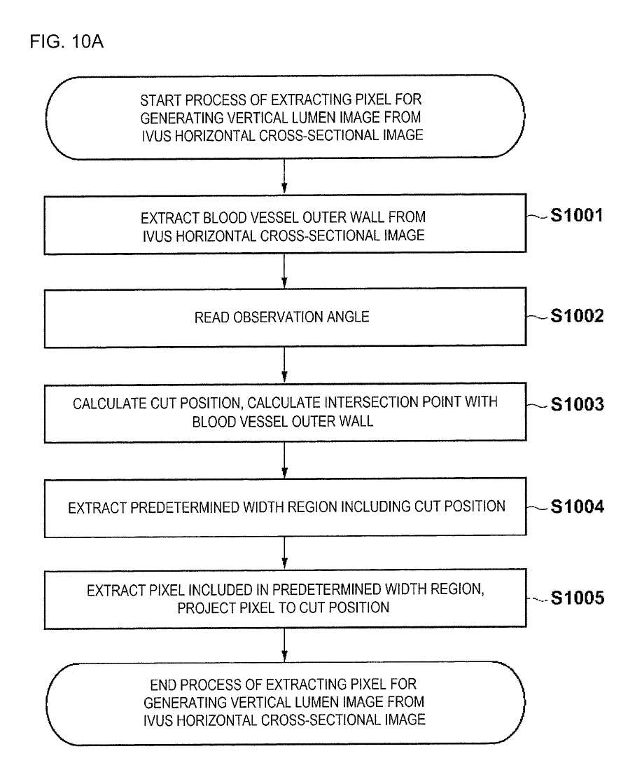

FIG. 10A is a flowchart illustrating flow in the process of extracting the pixel 706 for generating the vertical lumen image from the IVUS horizontal cross-sectional image 701. In Step S1001, the blood vessel outer wall is extracted from the IVUS horizontal cross-sectional image 701.

In Step S1002, an observation angle set in the vertical lumen image operation region 550 is read. In Step S1003, the cut position 703 is calculated based on the observation angle read in Step S1002. Furthermore, coordinates of the intersection point 704 with the blood vessel outer wall extracted in Step S1001 are calculated.

In Step S1004, the predetermined width region 705 including the cut position 703 is extracted. In Step S1005, a pixel included in the extracted predetermined width region is extracted, and is projected to the cut position 703, thereby extracting the pixel 706 at the cut position 703.

In accordance with an exemplary embodiment, FIG. 10B is a flowchart illustrating flow in the process of extracting the pixel 714 for generating the vertical lumen image from the OCT horizontal cross-sectional image 711. In Step S1011, the stent position is extracted from the OCT horizontal cross-sectional image 711.

In Step S1012, the observation angle set in the vertical lumen image operation region 550 is read. In Step S1013, the coordinates of the intersection point 704, which are calculated in Step S1003, are acquired.

In Step S1014, the semicircle region 713 including the stent position is calculated by causing the coordinates of the intersection point 704, which are acquired in Step S1013 to serve as end points. In Step S1015, a pixel included in the calculated semicircle region 713 is extracted, and is projected to the cut position 703, thereby extracting the pixel 714 at the cut position 703.

8. Example

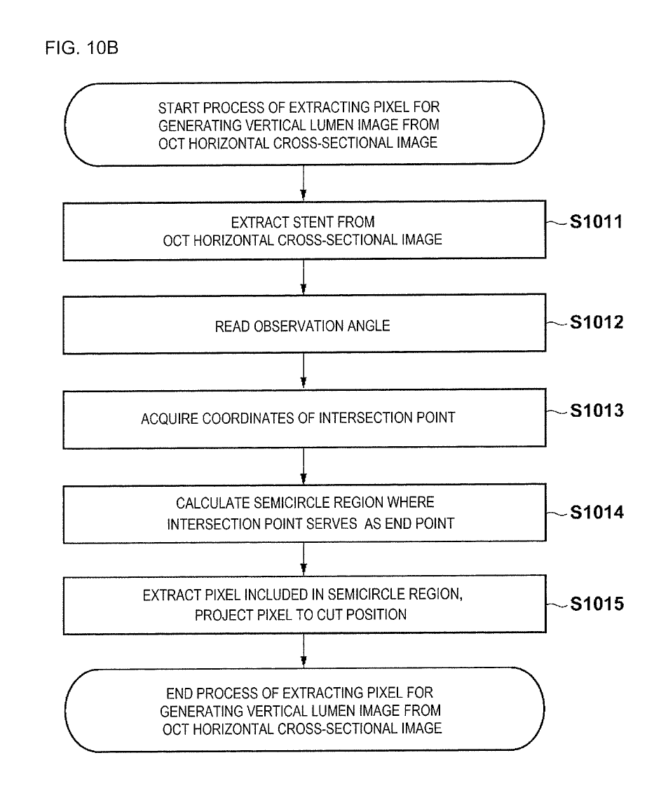

Next, an example of a generation process of a vertical lumen image (refer to FIG. 9) will be described. FIG. 11A illustrates a state where the vertical cross-sectional image is generated based on the pixel 706 extracted by performing Step S903 in the generation process of a vertical lumen image (refer to FIG. 9).

In addition, FIG. 11B is a view illustrating the vertical lumen image generated by performing a color allocation process by superimposing the pixel 714 extracted by performing Step S904 thereon. An example of FIG. 11 B illustrates a state where a white color is allocated to the portion of the stent and a black color is allocated to the bifurcated portion of the blood vessel.

As illustrated in FIG. 11B, according to the imaging apparatus for diagnosis 100 of the present embodiment, in a case of the further inner side from the blood vessel outer wall, a lumen surface of the blood vessel is displayed on the vertical cross-sectional image on a predetermined cut surface. Accordingly, for example a relationship between a position of a gap in the meshes of the stent and a position of the bifurcated portion of the blood vessel can be grasped or understood.

As is apparent from the above description, the imaging apparatus for diagnosis 100 according to the present embodiment adopts a configuration in which the IVUS vertical cross-sectional image, the OCT vertical cross-sectional image, and the vertical lumen image can be generated and displayed, based on the IVUS horizontal cross-sectional image and the OCT horizontal cross-sectional image, which can help enable a user to separately use the vertical cross-sectional image depending on respective uses.

In addition, a configuration is adopted so that the observation position can be changed on the vertical lumen image, which can help enable the user to observe the lumen surface using various observation angles. As a result, the usability of the vertical cross-sectional image can be expanded.

According to the first exemplary embodiment, the same process is performed on all of the horizontal cross-sectional images included in the predetermined range in the axial direction inside the body lumen. However, the present disclosure is not limited thereto. For example, a configuration may be adopted so that the process (refer to FIG. 10B) of extracting the pixel for generating the vertical lumen image with regard to a range from which the stent is extracted is performed on the OCT horizontal cross-sectional image.

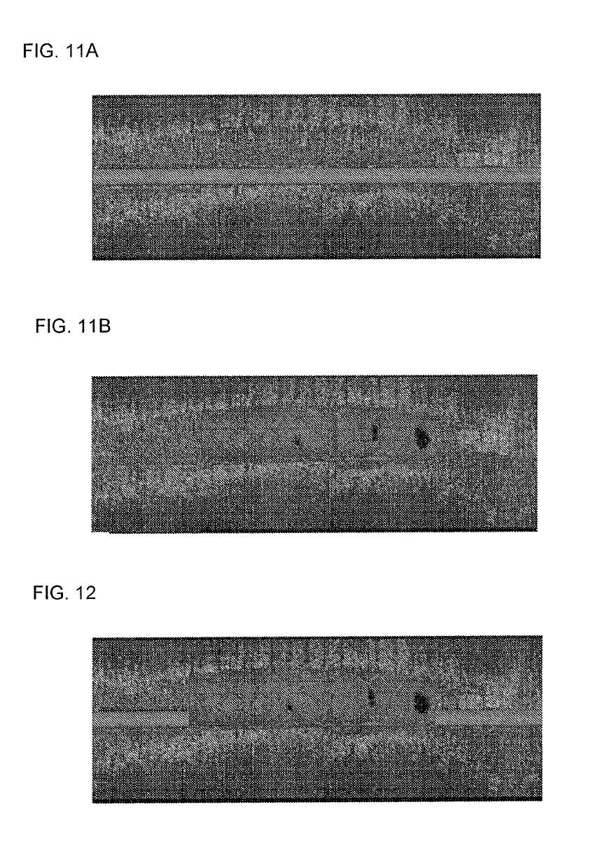

In this case, each pixel 706 for generating the vertical lumen image extracted from the IVUS horizontal cross-sectional image can be used with regard to a range from which the stent is not extracted in the axial direction within the vertical lumen image (refer to FIG. 12).

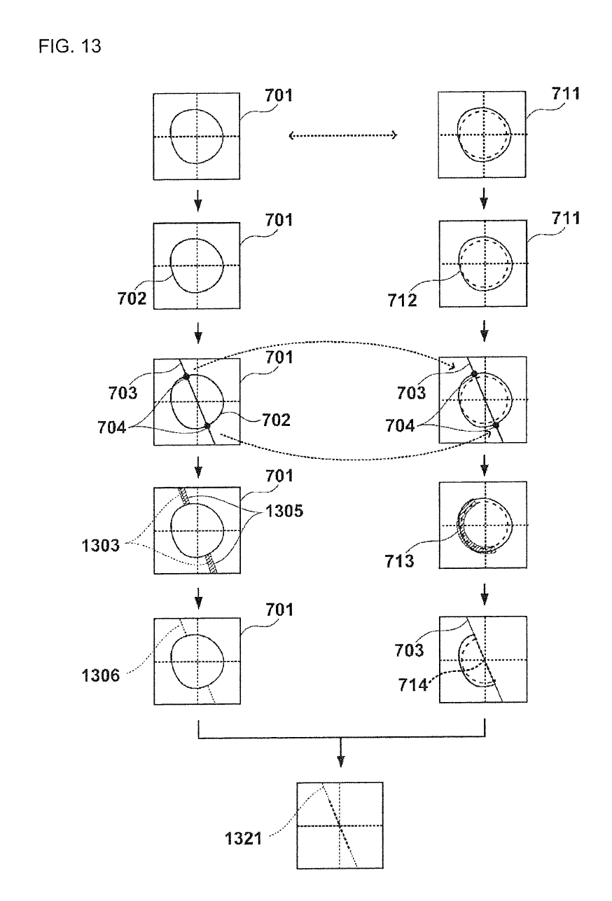

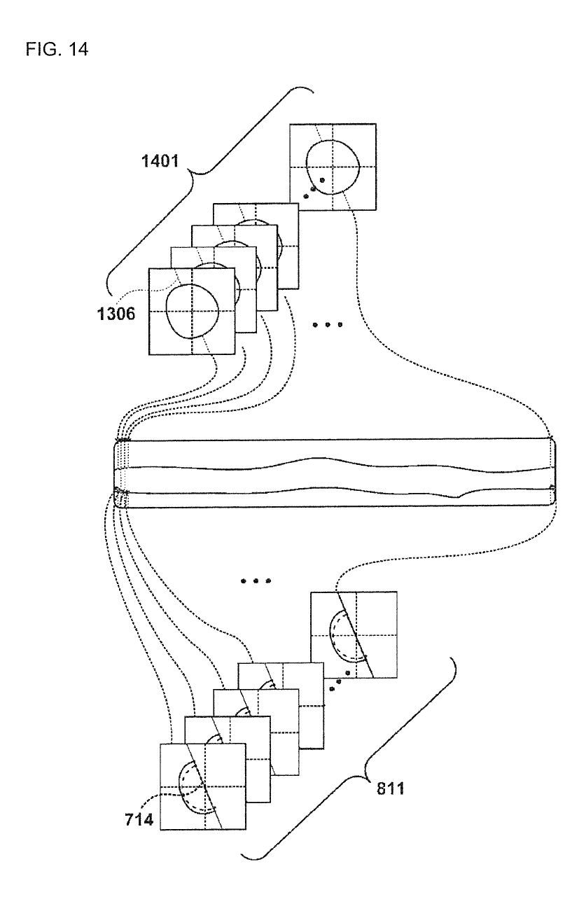

In addition, the above-described first exemplary embodiment adopts a configuration in which the pixel for generating the vertical lumen image which is extracted from the OCT horizontal cross-sectional image can be superimposed on the pixel for generating the vertical lumen image which is extracted from the IVUS horizontal cross-sectional image. However, the present disclosure is not limited thereto. For example, when extracting the pixel for generating the vertical lumen image from the IVUS horizontal cross-sectional image, a configuration may be made so as to extract the pixel by excluding the pixel between the intersection points 704 (refer to FIGS. 13 and 14). In this case, the pixel extracted from the IVUS horizontal cross-sectional image is connected to the pixel extracted from the OCT horizontal cross-sectional image at the intersection point position, thereby generating the vertical lumen image.

In addition, the above-described first embodiment adopts a configuration in which colors are allocated to the stent portion and the bifurcated portion of the blood vessel. However, the present disclosure is not limited thereto. The colors may be allocated to other portions as a target.

In addition, the above-described first embodiment adopts a configuration in which the vertical lumen image is displayed by also including the portion other than the stent portion in the axial direction inside the body lumen. However, the present disclosure is not limited thereto. A configuration may be adopted so as to extract and display only the stent portion.

In addition, the above-described first embodiment adopts a configuration in which based on the imaging apparatus for diagnosis comprising the IVUS function and the OCT function, the vertical lumen image is generated by using the IVUS horizontal cross-sectional image and the OCT horizontal cross-sectional image. However, the present disclosure is not limited thereto.

For example, a configuration may be adopted in which based on the imaging apparatus for diagnosis comprising two OCT functions, the vertical lumen image is generated by using two OCT horizontal cross-sectional images. Alternatively, a configuration may be adopted in which based on the imaging apparatus for diagnosis comprising two IVUS functions, the vertical lumen image is generated by using two IVUS horizontal cross-sectional images.

In addition, the above-described first embodiment adopts a configuration in which the “observation direction designation” button 553 is arranged in only the vertical lumen image operation region 550. However, the present disclosure is not limited thereto. For example, the same button may also be arranged in the vertical cross-sectional image operation region 540. Alternatively, a configuration may be adopted in which the OCT vertical cross-sectional image and the IVUS vertical cross-sectional image are updated in conjunction with the observation angle set by pressing down the “observation direction designation” button 553 arranged in the vertical lumen image operation region 550.

In addition, a configuration may be adopted in which an observation cross section can be optionally changed by displaying the observation cross section on the horizontal cross-sectional image, and by selecting and rotating the observation cross section using an instruction unit such as a mouse, a trackball or the like.

In addition, in the above-described first embodiment, the vertical lumen image including the stent is generated. However, the vertical lumen image excluding the stent may be generated.

The detailed description above describes imaging apparatus for diagnosis and an image processing method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An imaging apparatus for diagnosis which generates multiple first horizontal cross-sectional images and multiple second horizontal cross-sectional images which are cross-sectional images on a surface substantially orthogonal to an axial direction inside a body lumen, respectively in the axial direction by repeatedly transmitting and receiving a first signal and a second signal respectively while moving inside the body lumen in the axial direction, the apparatus comprising:
first extraction means for extracting a pixel corresponding to a cut position when a body lumen wall is cut by a plane substantially parallel to the axial direction, from the first horizontal cross-sectional image;
second extraction means for extracting a pixel corresponding to a lumen surface when the body lumen wall is cut by the plane, from the second horizontal cross-sectional image;
generation means for generating a vertical lumen image which is a lumen image when the body lumen wall is cut by the plane in such a way that a pixel obtained by projecting the pixel extracted by the second extraction means at the cut position is superimposed on the pixel extracted by the first extraction means; and
display means for displaying the vertical lumen image generated by the generation means.

2. The imaging apparatus for diagnosis according to claim 1, wherein the generation means generates the vertical lumen image by using the first horizontal cross-sectional image and the second horizontal cross-sectional image whose positions in the axial direction correspond to each other, within the multiple first horizontal cross-sectional images and the multiple second horizontal cross-sectional images.

3. The imaging apparatus for diagnosis according to claim 1, comprising:
setting means for setting an angle formed around the axis of the plane;
the first extraction means extracts a pixel corresponding to a cut position when the body lumen wall is cut by a plane substantially orthogonal to the angle set by the setting means; and
the second extraction means extracts a pixel corresponding to a lumen surface when the body lumen is cut by the plane substantially orthogonal to the angle set by the setting means.

4. The imaging apparatus for diagnosis according to claim 3, wherein the display means updates the display by using a vertical lumen image newly generated by the generation means, in response to a change in the angle set by the setting means.

5. The imaging apparatus for diagnosis according to claim 1, wherein the second extraction means further extracts a pixel corresponding to a cut position when the body lumen wall is cut by the plane;
the generation means further generates:
a first vertical cross-sectional image which is a cross-sectional image when the body lumen wall is cut by the plane, by using the pixel extracted by the first extraction means; and
a second vertical cross-sectional image which is a cross-sectional image when the body lumen wall is cut by the plane, by using the pixel which is extracted by the second extraction means and corresponds to the cut position when the body lumen wall is cut by the plane; and
the display means further displays the first vertical cross-sectional image and the second vertical cross-sectional image.

6. The imaging apparatus for diagnosis according to claim 1, wherein the first signal is an ultrasound signal, and the second signal is an optical signal.

7. The imaging apparatus for diagnosis according to claim 6, wherein the first extraction means extracts a pixel corresponding to a cut position when a blood vessel outer wall is cut by a plane substantially parallel to the axial direction, and the second extraction means extracts a pixel corresponding to a position of a stent when the blood vessel outer wall is cut by the plane.

8. The imaging apparatus for diagnosis according to claim 7, wherein the generation means generates the vertical lumen image by using the second horizontal cross-sectional image from which the stent is detected, among the multiple second horizontal cross-sectional images.

9. The imaging apparatus for diagnosis according to claim 7, comprising:
allocation means for allocating a color to the pixel corresponding to the position of the stent, within the generated vertical lumen image.

10. An image processing method of an imaging apparatus for diagnosis which generates multiple first horizontal cross-sectional images and multiple second horizontal cross-sectional images which are cross-sectional images on a surface substantially orthogonal to an axial direction inside a body lumen, respectively in the axial direction by repeatedly transmitting and receiving a first signal and a second signal respectively while moving inside the body lumen in the axial direction, the method comprising:
a first extraction step of extracting a pixel corresponding to a cut position when a body lumen wall is cut by a plane substantially parallel to the axial direction, from the first horizontal cross-sectional image;
a second extraction step of extracting a pixel corresponding to a lumen surface when the body lumen wall is cut by the plane, from the second horizontal cross-sectional image;
a generation step of generating a vertical lumen image which is a lumen image when the body lumen wall is cut by the plane in such a way that a pixel obtained by projecting the pixel extracted in the second extraction step at the cut position is superimposed on the pixel extracted in the first extraction step; and
a display step of displaying the vertical lumen image generated in the generation step.

11. The image processing method according to claim 10, comprising:
generating the vertical lumen image by using the first horizontal cross-sectional image and the second horizontal cross-sectional image whose positions in the axial direction correspond to each other, within the multiple first horizontal cross-sectional images and the multiple second horizontal cross-sectional images.

12. The image processing method according to claim 10, comprising:
a setting step for setting an angle formed around the axis of the plane;
the first extraction step extracts a pixel corresponding to a cut position when the body lumen wall is cut by a plane substantially orthogonal to the angle set by the setting means; and
the second extraction step extracts a pixel corresponding to a lumen surface when the body lumen is cut by the plane substantially orthogonal to the angle set by the setting means.

13. The image processing method according to claim 12, comprising:
updating the display step by using a vertical lumen image newly generated by the generation step, in response to a change in the angle set by the setting step.

14. The imaging apparatus for diagnosis according to claim 10, wherein the second extraction step further extracts a pixel corresponding to a cut position when the body lumen wall is cut by the plane;
the generation step further generates:
a first vertical cross-sectional image which is a cross-sectional image when the body lumen wall is cut by the plane, by using the pixel extracted by the first extraction step; and
a second vertical cross-sectional image which is a cross-sectional image when the body lumen wall is cut by the plane, by using the pixel which is extracted by the second extraction step and corresponds to the cut position when the body lumen wall is cut by the plane; and
the display step further displays the first vertical cross-sectional image and the second vertical cross-sectional image.

15. The image processing method according to claim 10, wherein the first signal is an ultrasound signal, and the second signal is an optical signal.

16. The image processing method according to claim 15, wherein the first extraction step extracts a pixel corresponding to a cut position when a blood vessel outer wall is cut by a plane substantially parallel to the axial direction, and the second extraction step extracts a pixel corresponding to a position of a stent when the blood vessel outer wall is cut by the plane; and
wherein the generation step generates the vertical lumen image by using the second horizontal cross-sectional image from which the stent is detected, among the multiple second horizontal cross-sectional images.

17. A non-transitory computer-readable recording medium with a program stored therein which causes a computer to execute each process of the image processing method according to claim 10.

18. An imaging apparatus for diagnosis which generates multiple first horizontal cross-sectional images and multiple second horizontal cross-sectional images which are cross-sectional images on a surface substantially orthogonal to an axial direction inside a body lumen, respectively in the axial direction by repeatedly transmitting and receiving a first signal and a second signal respectively while moving inside the body lumen in the axial direction, the apparatus comprising:
first generation means for generating a planar image of a body lumen wall at a cut position when the body lumen wall is cut by a plane substantially parallel to the axis direction inside the body lumen, based on the multiple first horizontal cross-sectional images;
second generation means for generating a three-dimensional image of a lumen surface of the body lumen which is interposed between the planar images, based on the multiple second horizontal cross-sectional images; and
synthesis display means for displaying by synthesizing the planar image and the three-dimensional image.

19. The imaging apparatus for diagnosis according to claim 18, wherein
the first signal is an ultrasound signal, and the second signal is an optical signal.

20. The imaging apparatus for diagnosis according to claim 18, wherein
the synthesis display means displays by synthesizing a projection image obtained by projecting the three-dimensional image at the cut position and the planar image.
